(19) United States
(12) Patent Application Publication
(10) Pub. No.: US 2023/0313032 A1
(43) Pub. Date: Oct. 5, 2023

(54) SENSITIZATION ENHANCEMENT OF SOLID-STATE PHOTONIC UPCONVERSION

(71) Applicants: The Regents of The University of Colorado, a body corporate, Denver, CO (US); Alliance for Sustainable Energy, LLC, Gloden, CO (US); Colorado School of Mines, Golden, CO (US)

(72) Inventors: Sean Shaheen, Boulder, CO (US); Daniel Weingarten, Boulder, CO (US); Michael LaCount, Davis, CA (US); Mark T. Lusk, Highlands Ranch, CO (US); Jao Van de Lagemaat, Arvada, CO (US); Garry Rumbles, Boulder, CO (US)

(21) Appl. No.: 18/022,203

(22) PCT Filed: Aug. 20, 2021
(86) PCT No.: PCT/US2021/046801

§ 371 Date: Feb. 20, 2023

Related U.S. Application Data
(60) Provisional application No. 63/068,358, filed on Aug. 20, 2020.

(51) Int. Cl. C09K 11/06; C08G 61/12; C08K 5/3417; C09B 67/20; C09B 67/22; C08J 5/18

(52) U.S. Cl. CPC C09K 11/06; C08G 61/12; C08K 5/3417; C09B 67/0064; C09B 67/0033; C08J 5/18; C09K 2211/1416; C09K 2211/1425; C09K 2211/1433; C08G 2261/124; C08G 2261/3142; C08G 2261/312; C08G 2261/52; C08J 2365/00

(57) ABSTRACT

Cooperative energy pooling systems based on polymeric acceptors are provided herein. These systems exhibit delayed excitation of the acceptor when excited at sensitizer absorption wavelengths, and displayed CEP occurring on a timescale of tens to hundreds of picoseconds.

### CROSS-REFERENCE TO RELATED APPLICATIONS

This application claims the benefit of priority to U.S. Provisional Application 63/068,358 filed Aug. 20, 2020, which is hereby incorporated herein by reference in its entirety.

### STATEMENT OF GOVERNMENT SUPPORT

This invention was made with government support under grant number CHE1125937, awarded by the National Science Foundation, and grant number DE-AC36-08GO28308, awarded by the U.S. Department of Energy. The government has certain rights in the invention.

### BACKGROUND

Upconversion materials have potential applications in a wide-range of fields, such as biosensing, chemical sensing, in vivo imaging, drug delivery, photodynamic therapy and photoactivation. Upconverting luminescence refers to an anti-Stokes type process in which the sequential absorption of two or more photons leads to the emission of light at shorter wavelength (e.g., ultraviolet, visible, and near-infrared) than the excitation wavelength. There is an ongoing need for new upconversion materials and improved methods of syntheses and applications thereof, especially those that require biocompatibility.

Cooperative energy pooling (CEP) is an energy transfer mechanism that provides an alternative route towards efficient and applicable photon upconversion. CEP is the process of two photoexcited sensitizer chromophores non-radiatively transferring their energy to a single higher-energy state in an acceptor chromophore. Theoretical work modelling three-body FRET processes in the late 1990s laid the foundation for a quantum electrodynamical understanding of the CEP process and more recent computational work has highlighted the dependence of the CEP process on both the separation distance and relative orientations of sensitizer and acceptor chromophores. There is a clear need for the development of systems having improved CEP yields over the previous systems.

### SUMMARY

In accordance with the purposes of the disclosed compositions, systems, and methods embodied and broadly described herein, the present disclosure provides compositions, systems, and methods based on polymer-based cooperative energy pooling (CEP) systems. Two distinct polymer-based CEP systems are exemplified herein, both of which presented improved CEP yields over Rhod6G/Stilb420 CEP system. Measurements of the internal quantum yield of CEP within the CEP systems are also provided. Femtosecond-scale transient absorption spectroscopy (TAS) data are also provided, displaying the CEP energy transfer process with time-resolution to clearly observe the energy transfer from sensitizers to acceptor.

In some examples, methods for enhancing upconversion luminescence of a solid phase composition comprising a multi-photon absorbing conjugated polymer and a sensitizer, wherein the conjugated polymer is separated from the sensitizer by an average distance of 5 nm or less are provided. The method can include irradiating the composition at a wavelength corresponding to the sensitizer absorption thereby generating a plurality of photoexcited sensitizers, allowing the plurality of photoexcited sensitizers to simultaneously transfer their energies to a higher-energy state on the conjugated polymer, wherein the emission spectrum of the photoexcited sensitizer at least partially overlaps with the multi-photon absorption spectrum of the conjugated polymer, such that there is resonant coupling between the sensitizer transition dipole and the conjugated polymer multi-photon tensor, and detecting luminescence in a spectral region characteristic of the conjugated polymer activated by the photoexcited sensitizers.

In some examples, the emission spectrum of the conjugated polymer exhibits negligible overlap with the absorption spectrum of the sensitizer.

In some examples, the multi-photon absorbing conjugated polymer can be a two-photon absorbing conjugated polymer. For example, the conjugated polymer can comprise a polyfluorene, a polyarylene, a polyphenylene, a polyanthracene, a polypyrene, a phenanthrene, a heterocyclic polyarylene such as a poly(thienylene), a poly(pyridine), an oxadiazole-containing polymer, a quinoline-containing polymer, a silole-containing polymer, a poly(3-alkyl-thiophene), or a combination thereof. In some instances, the conjugated polymer comprises a polyfluorene, such as a polyfluorene selected from the group consisting of:

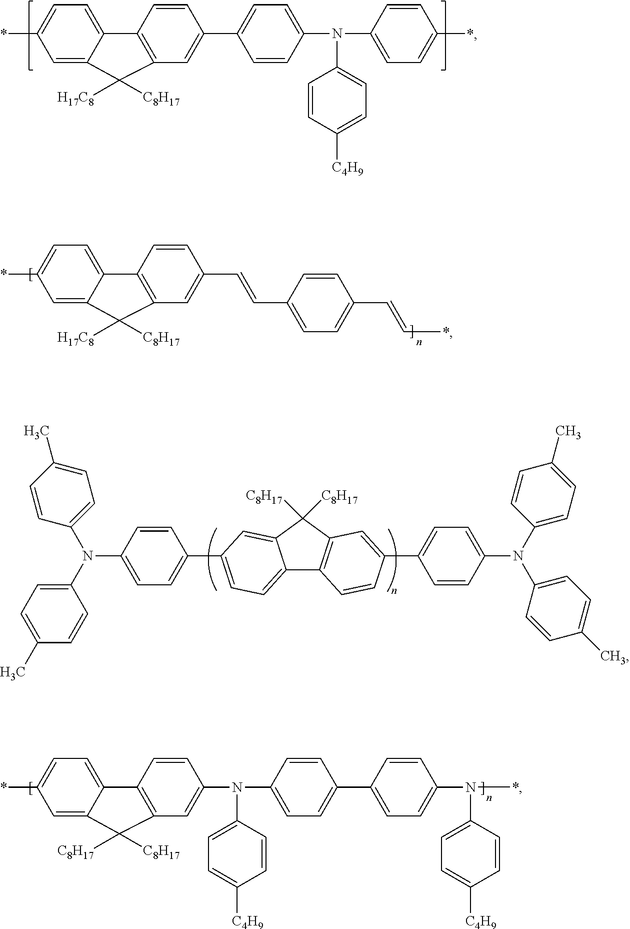

and combinations thereof.

The sensitizer can, for example, comprise a near infrared absorbing organic chromophore. In some examples, the sensitizer can comprise a cationic dye, an anionic dye, a nonionic dye, an amphoteric dye, a metal-ligand complex, fluorescein, chlorophyll, a phthalocyanine, an indodicarbocyanine, or a mixture thereof. In some examples, the sensitizer comprises zinc phthalocyanine (ZnPC) (structure shown below), 1,1,3,3,3,3-Hexamethyl-indodicarbocyanine iodide (HIDC) (structure shown below), or a combination thereof which structures are shown below.

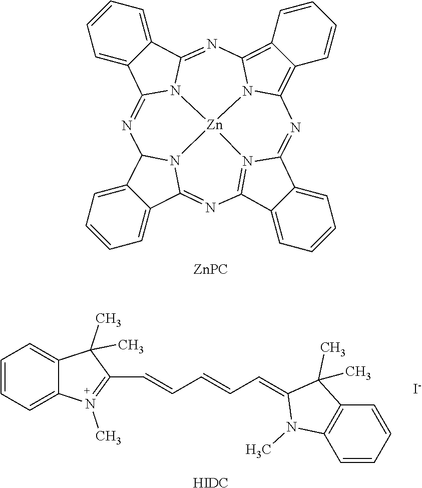

The amount of sensitizer and conjugated polymer in the composition can vary. For example, the molar ratio of the sensitizer to the conjugated polymer can be from 1:10 to 1:100 (e.g., from 1:20 to 1:60, or from 1:30 to 1:50). In some examples, the molar ratio of the sensitizer to the conjugated polymer can be 1:40.

As described herein, the composition comprising the conjugated polymer and sensitizer is a solid phase composition. The solid phase composition can be in the form of a nanofilm or a nanoparticle, e.g. to facilitate optimum sensitizer-acceptor separation distance for increasing the overall rate and yield of CEP upconversion.

In some examples, the composition is a nanofilm having an average thickness of 500 nm or less (e.g., 350 nm or less, or 300 nm or less). In some examples, the nanofilm has an average thickness of from 50 to 500 nm, from 100 to 300 nm, or from 200 to 300 nm.

In some examples, the composition comprises nanoparticles having an average particle size of 500 nm or less (e.g., 350 nm or less, or 300 nm or less). In some examples, the nanoparticles have an average particle size of from 50 to 500 nm, from 100 to 300 nm, or from 200 to 300 nm.

Systems for enhancing upconversion luminescence are also disclosed. In addition to the solid phase composition comprising the multi-photon absorbing conjugated polymer and the sensitizer, the systems can further include a source of radiation for irradiating the solid-phase composition at a wavelength corresponding to the sensitizer absorption.

For example, also disclosed herein are systems for enhancing upconversion luminescence comprising: a solid phase composition comprising a multi-photon absorbing conjugated polymer and a sensitizer, wherein the solid phase composition is in the form of a nanofilm or nanoparticles, and wherein the conjugated polymer is separated from the sensitizer by an average distance of 5 nm or less; wherein the emission spectrum of the sensitizer at least partially overlaps with the multi-photon absorption spectrum of the conjugated polymer, such that when the sensitizer becomes electronically excited, there is resonant coupling between the sensitizer transition dipole and the conjugated polymer multi-photon tensor; and a source of radiation for irradiating the composition at a wavelength corresponding to the sensitizer absorption.

In some examples of the systems, the sensitizer and the multi-photon absorbing conjugated polymer are in a molar ratio from 1:10 to 1:100, from 1:20 to 1:60, or from 1:30 to 1:50. In some examples of the systems, the sensitizer and the multi-photon absorbing conjugated polymer are in a molar ratio of 1:40.

In some examples of the systems, the multi-photon absorbing conjugated polymer is a two-photon absorbing conjugated polymer. In some examples of the systems, the emission spectrum of the conjugated polymer exhibits negligible overlap with the absorption spectrum of the sensitizer.

In some examples of the systems, the conjugated polymer comprises a polyfluorene, a polyarylene, a polyphenylene, a polyanthracene, a polypyrene, a phenanthrene, a heterocyclic polyarylene such as a poly(thienylene), a poly(pyridine), an oxadiazole-containing polymer, a quinoline-containing polymer, a silole-containing polymer, a poly(3-alkyl-thiophene), or a combination thereof. In some examples of the systems, the conjugated polymer comprises a polyfluorene. In some examples of the systems, the conjugated polymer comprises a polyfluorene selected from the group consisting of:

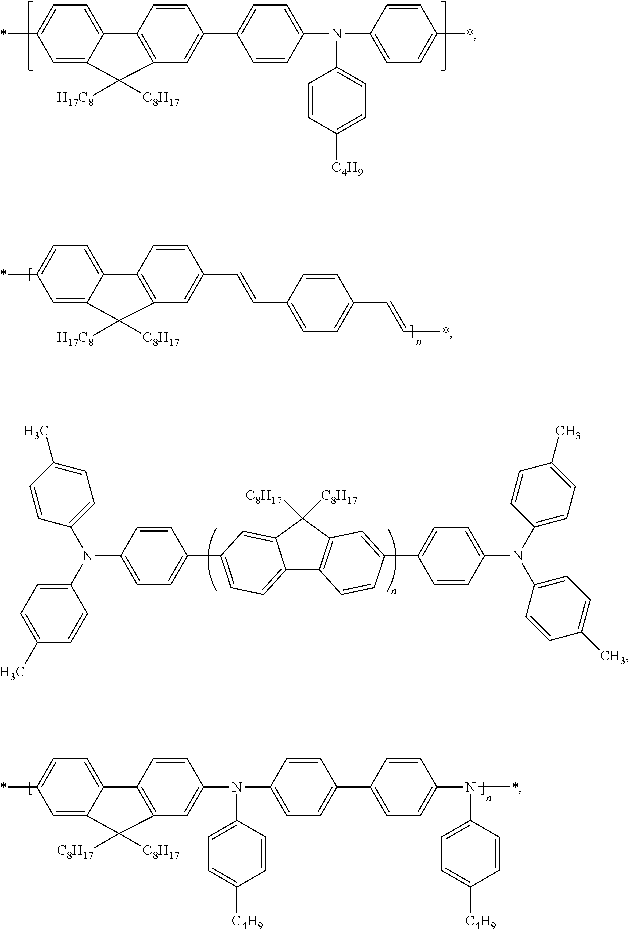

and combinations thereof.

In some examples of the systems, the sensitizer comprises a near infrared absorbing organic chromophore. In some examples of the systems, the sensitizer is selected from a cationic dye, an anionic dye, a nonionic dye, an amphoteric dye, a metal-ligand complex, fluorescein, chlorophyll, a phthalocyanine, an indodicarbocyanine, or a mixture thereof. In some examples of the systems, the sensitizer comprises:

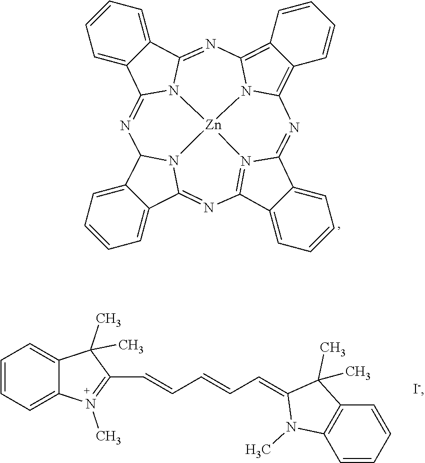

or a combination thereof.

In some examples of the systems, the composition is a nanofilm having an average thickness of 500 nm or less, 350 nm or less, or 300 nm or less. In some examples of the systems, the nanofilm has an average thickness of from 50 nm to 500 nm, from 100 nm to 300 nm, or 200 to 300 nm.

In some examples of the systems, the composition comprises nanoparticles having an average particle size of 500 nm or less, 350 nm or less, or 300 nm or less. In some examples of the systems, the nanoparticles have an average particle size of from 50 nm to 500 nm, from 100 nm to 300 nm, or from 200 to 300 nm.

Also disclosed herein are compositions for enhancing upconversion luminescence, the compositions comprising: a solid phase composition comprising multi-photon absorbing conjugated polymer and a sensitizer, wherein the solid phase composition is in the form of a nanofilm or nanoparticles, and wherein the conjugated polymer is separated from the sensitizer by an average distance of 5 nm or less; wherein the emission spectrum of the sensitizer at least partially overlaps with the multi-photon absorption spectrum of the conjugated polymer, such that when the sensitizer becomes electronically excited, there is resonant coupling between the sensitizer transition dipole and the conjugated polymer multi-photon tensor; and wherein the sensitizer and the multi-photon absorbing conjugated polymer are in a molar ratio from 1:10 to 1:100, from 1:20 to 1:60, or from 1:30 to 1:50. In some examples of the compositions, the sensitizer and the multi-photon absorbing conjugated polymer are in a molar ratio of 1:40.

In some examples of the compositions, the multi-photon absorbing conjugated polymer is a two-photon absorbing conjugated polymer. In some examples of the compositions, the emission spectrum of the conjugated polymer exhibits negligible overlap with the absorption spectrum of the sensitizer.

In some examples of the compositions, the conjugated polymer comprises a polyfluorene, a polyarylene, a polyphenylene, a polyanthracene, a polypyrene, a phenanthrene, a heterocyclic polyarylene such as a poly(thienylene), a poly(pyridine), an oxadiazole-containing polymer, a quinoline-containing polymer, a silole-containing polymer, a poly(3-alkyl-thiophene), or a combination thereof. In some examples of the compositions, the conjugated polymer comprises a polyfluorene. In some examples of the compositions, the conjugated polymer comprises a polyfluorene selected from the group consisting of:

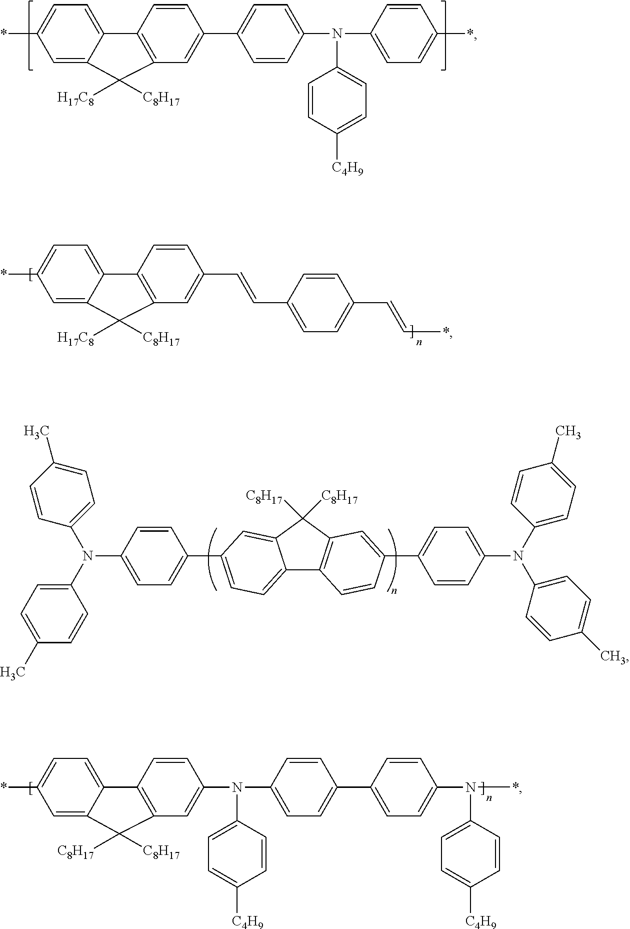

and combinations thereof.

In some examples of the compositions, the sensitizer comprises a near infrared absorbing organic chromophore. In some examples of the compositions, the sensitizer is selected from a cationic dye, an anionic dye, a nonionic dye, an amphoteric dye, a metal-ligand complex, fluorescein, chlorophyll, a phthalocyanine, an indodicarbocyanine, or a mixture thereof. In some examples of the compositions, the sensitizer comprises:

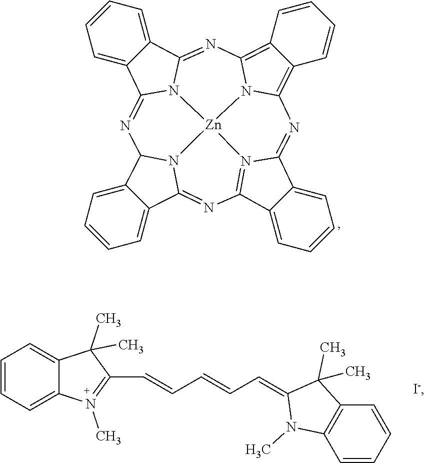

or a combination thereof.

In some examples of the compositions, the composition is a nanofilm having an average thickness of 500 nm or less, 350 nm or less, or 300 nm or less. In some examples of the compositions, the nanofilm has an average thickness of from 50 nm to 500 nm, from 100 nm to 300 nm, or from 200 to 300 nm.

In some examples of the compositions, the composition comprises nanoparticles having an average particle size of 500 nm or less, 350 nm or less, or 300 nm or less. In some examples of the compositions, the nanoparticles have an average particle size of from 50 nm to 500 nm, from 100 nm to 300 nm, or from 200 to 300 nm.

The composition, methods, and systems described herein has applications in fields including biomedical imaging, biomedical therapeutics and cancer treatments, optical communications, optical computing, and solar energy conversion. Accordingly, imaging methods comprising, administering to a subject a composition as described herein, irradiating the composition at a wavelength corresponding to the sensitizer absorption, and detecting luminescence in a spectral region characteristic of the conjugated polymer activated by the plurality of photoexcited sensitizers are provided. Optoelectronic signaling devices comprising a composition as described herein, preferably wherein the device is for optical communication, optical computing, or solar energy conversion are also provided.

The foregoing and other features of the disclosure will become apparent from the following detailed description of several embodiments which proceed with reference to the accompanying drawings.

### BRIEF DESCRIPTION OF THE DRAWINGS

The accompanying figures, which are incorporated in and constitute a part of this specification, illustrate several aspects of the disclosure, and together with the description, serve to explain the principles of the disclosure.

FIG. 1A shows the molecular structure of ADS259BE.

FIG. 1B shows the molecular structure of ADS128GE.

FIG. 1C shows the molecular structure of ADS329BE.

FIG. 1D shows the molecular structure of ADS251BE.

FIG. 1E shows the molecular structure of zinc phthalocyanine.

FIG. 1F shows the molecular structure of HIDC iodide.

FIG. 2A is a graph showing the spectral properties of ZnPC/ADS128 CEP system. Normalized absorption, emission, and upconverted emission spectra of ZnPC/ADS128 blend film. Upconverted emission was measured under excitation at 677 nm. The emission spectrum is cut off due to the use of a 500 nm shortpass filter to prevent scattered excitation light from contaminating the upconverted signal. Normal emission was measured using 330 nm excitation light.

FIG. 2B is a graph showing the spectral properties of ZnPC/ADS128 CEP system. Normalized absorption and emission spectra of ADS128 (acceptor) and ZnPC (sensitizer) in pristine solutions with THF solvent. Steady-state emission spectra were measured under excitation at 350 nm.

FIG. 3A is a graph showing the spectral properties of HIDC/ADS259 CEP system. Normalized absorption, emission, and upconverted emission spectra of HIDC/ADS259 blend film. Upconverted emission was measured under excitation at 664 nm. The emission spectrum is cut off due to the use of a 500 nm shortpass filter to prevent scattered excitation light from contaminating the upconverted signal. Normal emission was measured using 390 nm excitation light. Magnified red emission peak measured under excitation at 590 nm to target sensitizer absorption.

FIG. 3B is a graph showing the spectral properties of HIDC/ADS259 CEP system. Normalized absorption and emission spectra of ADS259 (acceptor) and HIDC (sensitizer) in pristine solutions with THF solvent. Steady-state emission spectra were measured under excitation at 330 nm and 520 nm for ADS259 and HIDC, respectively.

FIG. 4A is a graph showing the excitation dependence of ZnPC/ADS128 blend films. The squares indicate the measured upconverted emission at 486 nm from ZnPC/ADS128 blend films as a function of 677 nm excitation intensity plotted on a log-log scale. The indicated lines are quadratic and linear fits to the first and last three data points, respectively. The black circles represent the instantaneous power-law dependence of the measured excitation dependence as determined by the slope of a linear fit to a sliding boxcar window of eight points on the log-log plot of the excitation dependence data.

FIG. 4B is a graph showing the excitation dependence of HIDC/ADS259 blend films. The squares indicate the measured upconverted emission at 436 nm from HIDC/ADS259 blend films as a function of 664 nm excitation intensity plotted on a log-log scale. The indicated lines are quadratic and linear fits to the first and last three data points, respectively. The black circles represent the instantaneous power-law dependence of the measured excitation dependence as determined by the slope of a linear fit to a sliding boxcar window of eight points on the log-log plot of the excitation dependence data.

FIG. 5A is a graphs showing TA spectra of pristine ADS128 thin films. When excited at 400 nm the ADS128 polymer displays two noticeable features at 469 nm and 515 nm with a slight should feature at 555 nm, all with −Δ OD peaks. The 515 nm peak and the shoulder feature are nearly absent after 150 ps, indicating that the different features each have distinct lifetimes.

FIG. 5B is a graph showing TA spectra of pristine ZnPC thin films. When excited at 677 nm the ZnPC sensitizer displays a main +Δ OD plateau feature stretching between ˜430-600 nm, with slight sub-features at 485 nm, 530 nm, and 596 nm. Since the shape of the spectrum changes over time the different features must have slightly different lifetimes, but the data was too noisy for accurate fitting of the distinct decay lifetimes. The overall lifetime of the ZnPC excited state is noticeably longer than the ADS128 excited state lifetimes.

FIG. 6A is a graph showing the TA spectra of ADS128/ZnPC CEP film. TA spectra for the ADS128/ZnPC thin film under excitation at 677 nm. The main plateau feature of the ZnPC sensitizer and the −ΔOD peak at 469 nm of the ADS128 acceptor are both present, with the ADS128 feature rising at a later time than the ZnPC feature. This delayed rise indicates energy transfer from ZnPC to ADS128 and hence CEP, as discussed in detail in the text. The other sub-features of both chromophores are absent. Note that the overall signal is much lower in intensity than the previous TA spectra, largely due to the superposition of two signals with opposite ΔOD. The data was binomially smoothed in five passes in order to reduce some of the noise inherent in such a low-strength signal.

FIG. 6B is a graph showing the TA spectra of ADS128/ZnPC CEP film. Kinetic traces corresponding at the wavelengths of the component chromophore features are displayed on a normalized ΔOD axis to facilitate comparison of rise-times and decay times. The previously distinct features above 485 nm all appear with similar decay times. However, the kinetics at 469 nm show a delayed rise to +ΔOD corresponding to the ZnPC excited stat, and subsequent a decay to −ΔOD corresponding to a delayed rise of the ADS128 excited state and indicating CEP.

### DETAILED DESCRIPTION

The following description of the disclosure is provided as an enable teaching of the disclosure in its best, currently known embodiments. To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are part of the present disclosure. Thus, the following description is provided as illustrative of the principles of the present disclosure and not in limitation thereof.

### Definitions

Unless defined otherwise, all technical and scientific terms used herein have the same meaning as commonly understood to one of ordinary skill in the art to which this invention belongs. The following definitions are provided for the full understanding of terms used in the specification.

Throughout the description and claims of this specification the word “comprise” and other forms of the word, such as “comprising” and “comprises,” means including but not limited to, and is not intended to exclude, for example, other additives, components, integers, or steps.

As used in the specification and claims, the singular form “a”, “an”, and “the” include plural references unless the context clearly dictates otherwise. For example, the term “an anionic dye” includes a plurality of anionic dyes, including mixtures thereof.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed.

Values can be expressed herein as an “average” value. “Average” generally refers to the statistical mean value.

By “substantially” is meant within 5%, e.g., within 4%, 3%, 2%, or 1%.

“Exemplary” means “an example of” and is not intended to convey an indication of a preferred or ideal embodiment. “Such as” is not used in a restrictive sense, but for explanatory purposes.

It is understood that throughout this specification the identifiers “first” and “second” are used solely to aid in distinguishing the various components and steps of the disclosed subject matter. The identifiers “first” and “second” are not intended to imply any particular order, amount, preference, or importance to the components or steps modified by these terms.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human. Thus, the subject can be a human or veterinary patient. The term “patient” refers to a subject under the treatment of a clinician, e.g., physician.

As used herein, “molecular weight” refers to number average molecular weight as measured by ¹H NMR spectroscopy, unless indicated otherwise.

“Polymer” means a material formed by polymerizing one or more monomers.

The term “(co)polymer” includes homopolymers, copolymers, or mixtures thereof.

The term “(meth)acryl . . . ” includes “acryl . . . ,” “methacryl . . . ,” or mixtures thereof.

As used herein, “biocompatible” describes a material that elicits an appropriate host response without any adverse effects, and is compatible with living cells, tissues, organs, or systems, and posing no risk of injury, toxicity, or rejection by the immune system.

As used herein, “upconversion” refers to a process in which the sequential absorption of two or more photons leads to the emission of light at shorter wavelength than the excitation wavelength.

As used herein, “sensitizer” refers to a molecule that absorbs energy (such as infrared energy) and transfers this energy non-radiatively to the activator.

As used herein, “activator” refers to a molecule which receives energy from the sensitizer and as a consequence thereof emits upconversion luminescence.

### Compositions and Systems

The present inventors have observed singlet-based cooperative energy pooling (CEP) upconversion in solid-state, air-exposed organic chromophore blends. CEP is the process of two photoexcited sensitizer chromophores non-radiatively transferring their energy to a single higher-energy state in an acceptor chromophore. CEP is carried out via a coupling of the emissive states of both sensitizers with the multi-photon absorption tensor of the acceptor. In this way the sensitizers act as photon energy storage centers that relax the stringent temporal and spatial constraints for achieving multi-photon absorption in the acceptor, enabling upconversion with greater efficiency and at reduced excitation intensities.

Optimization of sensitizer-acceptor pairs have shown that anomalously long lifetime of the CEP-excited state can be in part attributed to morphological selectivity of the CEP process. For instance, CEP is more likely to occur when there is a minimal separation distance between the sensitizers and the acceptor, and hence CEP energy transfer is likely to preferentially occur to acceptor chromophores whose nearest neighbors are sensitizers rather than other acceptors. This isolation from other acceptors then potentially extends the lifetime of the CEP-excited state towards its inherent radiative lifetime by reducing pathways for non-radiative decay via self-quenching. Thus, heterogeneity of the CEP composition morphology plays important role in the CEP rates and excited state lifetimes.

Accordingly, the present disclosure provides compositions and systems based on polymer-based cooperative energy pooling (CEP) systems. The polymer-based CEP systems provided herein exhibit improved CEP over the previous generation CEP system as these systems have larger acceptor multi-photon absorption cross-sections that extends to longer wavelengths, which provide improved spectral overlap between acceptor and sensitizer, and reduced FRET energy loss pathways, all of which are factors that are expected to improve CEP rates.

The compositions and systems herein comprise a polymer and a sensitizer. The polymers used in the present compositions and systems are multi-photon absorption conjugated polymers. The multi-photon absorption spectrum of the conjugated polymer at least partially overlaps with the emission spectrum of the sensitizer, such that when the sensitizer becomes electronically excited, there is resonant coupling between the sensitizer transition dipole and the conjugated polymer multi-photon tensor. The multi-photon absorption spectrum refers to an absorption spectrum of an excited electronic state of a molecule (the conjugated polymer in this case) after the absorption of at least two photons of identical or different frequencies in order to excite the molecule from one state (usually the ground state) to a higher energy.

As described herein, the multi-photon absorption spectrum of the conjugated polymer at least partially overlaps with the emission spectrum of the sensitizer. In some examples, the multi-photon absorption spectrum of the conjugated polymer can overlap with the emission spectrum of the sensitizer by 10% or more (e.g., 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, or 95% or more). In some examples, the multi-photon absorption spectrum of the conjugated polymer can overlap with the emission spectrum of the sensitizer by 100% or less (e.g., 95% or less, 90% or less, 85% or less, 80% or less, 75% or less, 70% or less, 65% or less, 60% or less, 55% or less, 50% or less, 45% or less, 40% or less, 35% or less, 30% or less, 25% or less, 20% or less, or 15% or less). The amount of overlap between the multi-photon absorption spectrum of the conjugated polymer and the emission spectrum of the sensitizer can range from any of the minimum values described above to any of the maximum values described above. For example, the multi-photon absorption spectrum of the conjugated polymer can overlap with the emission spectrum of the sensitizer by 10%-100% (e.g., from 10% to 45%, from 45% to 100%, from 10% to 40%, from 40% to 70%, from 70% to 100%, from 15% to 100%, from 10% to 95%, from 15% to 95%, or from 10% to 75%).

In some examples, the emission spectrum of the conjugated polymer exhibits negligible to virtually no observable overlap with the absorption spectrum of the sensitizer. For instance, the emission spectrum of the conjugated polymer overlaps with the absorption spectrum of the sensitizer by 10% or less (e.g., 9% or less, 8% or less, 7% or less, 6%, 5%, 4% or less, 3% or less, 2% or less, or 1% or less).

In some examples, the multi-photon absorbing conjugated polymer can be a two-photon absorbing conjugated polymer. In some examples, the conjugated polymer can comprise a polyfluorene, a polyarylene, a polyphenylene, a polyanthracene, a polypyrene, a phenanthrene, a heterocyclic polyarylene such as a poly(thienylene), a poly(pyridine), an oxadiazole-containing polymer, a quinoline-containing polymer, a silole-containing polymer, a poly(3-alkyl-thiophene), or a combination thereof. Other suitable conjugated polymers include, but are not limited to, a pyrrolyl, furanyl, imidazolyl, triazolyl, isoxazolyl, oxadiazolyl, furazanyl, pyridazinyl, pyrimidyl, pyrazinyl, triazinyl, tetrazinyl, benzofuranyl, benzothiophenyl, indolyl, isoindazolyl, benzimidazolyl, benzotriazolyl, benzoxazolyl, isoquinolyl, cinnolyl, quinazolyl, naphthyridyl, phthalazyl, phentriazyl, benzotetrazyl, carbazolyl, dibenzofuranyl, dibenzothiophenyl, acridyl, phenazyl, and combinations thereof.

In some examples, the conjugated polymer comprises a polyfluorene, such as a polyfluorene selected from the group consisting of:

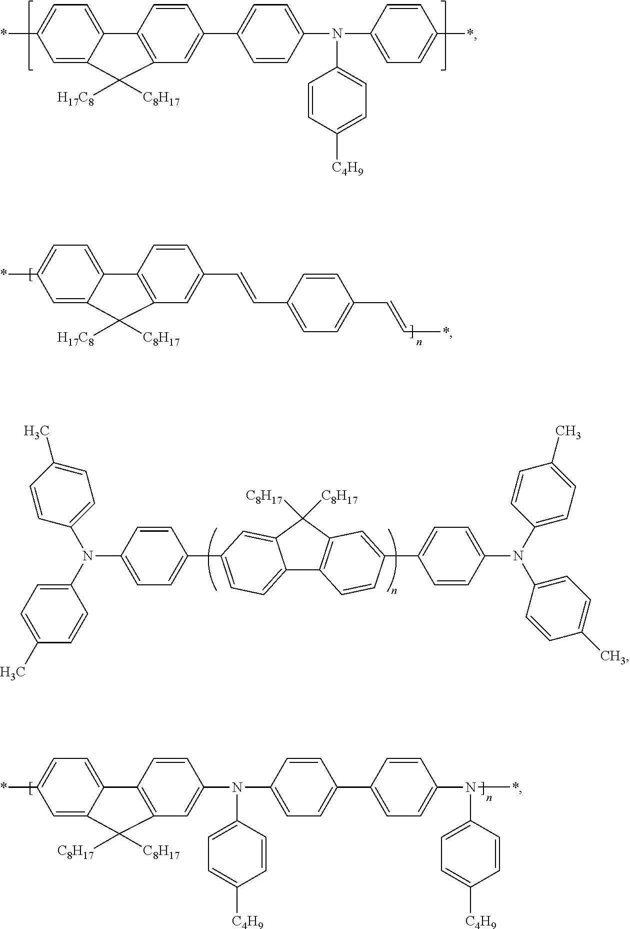

and combinations thereof.

As discussed herein, the multi-photon absorption spectrum of the conjugated polymer at least partially overlaps with the emission spectrum of the sensitizer. In some examples, the multi-photon absorption spectrum of the conjugated polymer can overlap significantly with the emission spectrum of the sensitizer. This allows efficient coupling between the sensitizer transition dipole and the conjugated polymer's multi-photon absorption tensor and hence a large CEP rate. Further, the use of sensitizers with high quantum yield even when aggregated can directly increase both the CEP rate but also the CEP radius.

The sensitizer can, for example, comprise a near infrared absorbing organic chromophore. For example, the sensitizer can comprise a cationic dye, an anionic dye, a nonionic dye, an amphoteric dye, a metal-ligand complex, fluorescein, chlorophyll, a phthalocyanine, an indodicarbocyanine, or a mixture thereof. Representative examples of suitable sensitizers include zinc phthalocyanine (ZnPC) and 1,1,3,3,3,3-Hexamethyl-indodicarbocyanine iodide (HIDC), the structures of which are shown below. In some examples, the sensitizer can include ZnPC, HIDC, or a combination thereof.

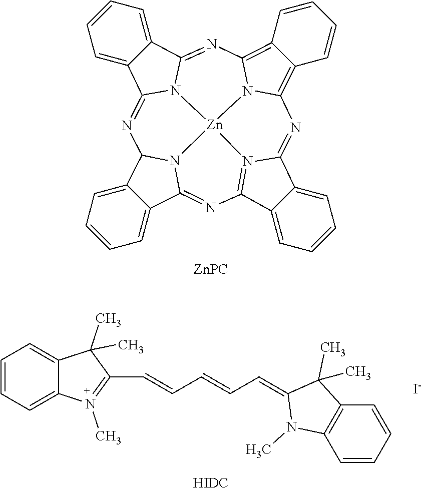

When sensitized by the sensitizer, photons are primarily transferred to either acceptors (i.e., the conjugated polymer) or neighboring sensitizers. Consequently, photons will either be transferred to an activator leading to upconversion and resultant luminescence emission, or alternatively encounter a quencher. Where the sensitizer concentration exceeds a certain amount, the chance of sensitized photons encountering quenchers is also increased thereby contributing to concentration quenching. In some examples, the sensitizer is a low self-quenching chromophore which leads to improved CEP radius, increased overall absorbance of the blend film, and increased exciton diffusivity, all of which improve overall CEP yields. One aspect of the CEP systems described herein is providing a blend of sensitizer and polymer such that the average sensitizer chromophores are isolated from other sensitizers.

For example, the amount of sensitizer and conjugated polymer can be present in a molar ratio of the sensitizer to the conjugated polymer of 1:10 or more (e.g., 1:20 or more, 1:30 or more, 1:40 or more, 1:50 or more, 1:60 or more, 1:70 or more, 1:80 or more, or 1:90 or more). In some examples, the amount of sensitizer and conjugated polymer can be present in a molar ratio of the sensitizer to the conjugated polymer of 1:100 or less (e.g., 1:90 or less, 1:80 or less, 1:70 or less, 1:60 or less, 1:50 or less, 1:40 or less, 1:30 or less, or 1:20 or less). The molar ratio of the sensitizer to the conjugated polymer can range from any of the minimum values described above to any of the maximum values described above. For example, the amount of sensitizer and conjugated polymer can be present in a molar ratio of the sensitizer to the conjugated polymer of from 1:10 to 1:100 (e.g., from 1:10 to 1:45, from 1:45 to 1:100, from 1:10 to 1:40, from 1:40 to 1:70, from 1:70 to 1:100, from 1:15 to 1:100, from 1:10 to 1:95, from 1:15 to 1:95, from 1:10 to 1:80, from 1:10 to 1:70, from 1:20 to 1:60, or from 1:30 to 1:50). In some examples, the amount of sensitizer and conjugated polymer can be present in a molar ratio of the sensitizer to the conjugated polymer of 1:40.

As described herein, the composition comprising the conjugated polymer and sensitizer is a solid phase composition. A solid phase composition provides several advantages as the solid matrix prevents or minimizes collisional quenching of the sensitizer and reduces solvent effects. The solid phase also provides a relatively rigid environment conducive to long emission lifetime and high luminescence efficiency. The solid phase composition can be in the form of a nanofilm or a nanoparticle to facilitate optimum sensitizer-acceptor separation distance for increasing the overall rate and yield of CEP upconversion. The shape of the nanoparticles can vary. In some examples, the nanoparticles can include spherical particles, non-spherical particles (such as elongated particles, cylindrical particles, rod-like particles, or any irregularly shaped particles), or combinations thereof.

As demonstrated in the examples, the approximate sensitizer-acceptor chromophore separation distance can be calculated. Adjustments to the nanofilm or nanoparticle morphology can reduce the separation distance of the sensitizer-acceptor chromophore and increase the overall rate and yield of CEP upconversion.

In some examples, the sensitizer is separated from the acceptor (conjugated polymer) by an average distance of no more than 5 nm (e.g., 5 nm or less, 4.5 nm or less, 4 nm or less, 3.5 nm or less, 3 nm or less, or 2.5 nm or less).

In some examples, the nanofilms can have an average thickness of 1 micrometer (μm, micron) or less (e.g., 750 nanometers (nm) or less, 500 nm or less, 400 nm or less, 300 nm or less, 250 nm or less, 200 nm or less, 175 nm or less, 150 nm or less, 125 nm or less, 100 nm or less, 90 nm or less, 80 nm or less, 70 nm or less, 60 nm or less, 50 nm or less, 45 nm or less, 40 nm or less, 35 nm or less, 30 nm or less, 25 nm or less, 15 nm or less, 10 nm or less, or 5 nm or less). In some examples, the nanofilms can have an average thickness of 1 nanometer (nm) or more (e.g., 2 nm or more, 3 nm or more, 4 nm or more, 5 nm or more, 10 nm or more, 15 nm or more, or 25 nm or more, 30 nm or more, 35 nm or more, 40 nm or more, 45 nm or more, 50 nm or more, 60 nm or more, 70 nm or more, 80 nm or more, 90 nm or more, 100 nm or more, 125 nm or more, 150 nm or more, 175 nm or more, 200 nm or more, 250 nm or more, 300 nm or more, 400 nm or more, 500 nm or more, or 750 nm or more). The nanofilms can have an average thickness ranging from any of the minimum values described above to any of the maximum values described above. For example, in certain examples, the nanofilms can have an average thickness of from 1 nm to 1000 nm (e.g., from 1 nm to 500 nm, from 500 nm to 1000 nm, from 1 nm to 200 nm, from 200 nm to 400 nm, from 400 nm to 600 nm, from 600 nm to 800 nm, from 800 nm to 1000 nm, from 5 nm to 1000 nm, from 1 nm to 900 nm, from 5 nm to 900 nm, from 1 nm to 750 nm, from 5 nm to 500 nm, from 50 to 500 nm, from 100 nm to 500 nm, from 100 nm to 350 nm, from 150 nm to 300 nm, from 100 to 300 nm, from 50 nm to 300 nm, from 200 nm to 350 nm, or from 200 nm to 300 nm).

The nanoparticles can have an average particle size. “Average particle size” and “mean particle size” are used interchangeably herein, and generally refer to the statistical mean particle size of the particles in a population of particles. For example, the average particle size for a plurality of particles with a substantially spherical shape can comprise the average diameter of the plurality of particles. For a particle with a substantially spherical shape, the diameter of a particle can refer, for example, to the hydrodynamic diameter. As used herein, the hydrodynamic diameter of a particle can refer to the largest linear distance between two points on the surface of the particle. Mean particle size can be measured using methods known in the art, such as evaluation by microscopy (e.g. electron microscopy) and/or dynamic light scattering.

In some examples, the nanoparticles can have an average particle size of 1 micron or less (e.g., 750 nm or less, 500 nm or less, 400 nm or less, 300 nm or less, 250 nm or less, 200 nm or less, 175 nm or less, 150 nm or less, 125 nm or less, 100 nm or less, 90 nm or less, 80 nm or less, 70 nm or less, 60 nm or less, 50 nm or less, 45 nm or less, 40 nm or less, 35 nm or less, 30 nm or less, 25 nm or less, 20 nm or less, 15 nm or less, 10 nm or less, or 5 nm or less). In some examples, the nanoparticles can have an average particle size of 1 nm or more (e.g., 2 nm or more, 3 nm or more, 4 or more, 5 nm or more, 10 nm or more, 15 nm or more, 25 nm or more, 30 nm or more, 35 nm or more, 40 nm or more, 45 nm or more, 50 nm or more, 60 nm or more, 70 nm or more, 80 nm or more, 90 nm or more, 100 nm or more, 125 nm or more, 150 nm or more, 175 nm or more, 200 nm or more, 250 nm or more, 300 nm or more, 400 nm or more, 500 nm or more, or 750 nm or more). The nanoparticles can have an average particle size ranging from any of the minimum values described above to any of the maximum values described above. For example, in certain examples, the nanoparticles can have an average particle size of from 1 nm to 1000 nm (e.g., from 1 nm to 500 nm, from 500 nm to 1000 nm, from 1 nm to 200 nm, from 200 nm to 400 nm, from 400 nm to 600 nm, from 600 nm to 800 nm, from 800 nm to 1000 nm, from 5 nm to 1000 nm, from 1 nm to 900 nm, from 5 nm to 900 nm, from 1 nm to 750 nm, from 5 nm to 500 nm, from 50 nm to 500 nm, from 100 nm to 500 nm, from 100 nm to 350 nm, from 100 nm to 300 nm, from 150 nm to 300 nm, from 50 nm to 300 nm, from 200 nm to 350 nm, or from 200 nm to 300 nm).

Systems for enhancing upconversion luminescence are also disclosed herein. In addition to the solid phase composition comprising a multi-photon absorbing conjugated polymer and a sensitizer, the systems can further include a source of radiation for irradiating the solid-phase composition at a wavelength corresponding to the sensitizer absorption. The source of radiation will depend on the particular sensitizer-acceptor chromophores used. In some examples, the sensitizer-acceptor chromophores can be a NIR-to-visible upconversion fluorescent composition. In this instance, the source of excitation can be NIR. The means for delivery of the source of radiation to the system can be, for example, via optical fibers, endoscopes, external light, and external laser.

Articles comprising the upconversion compositions are also disclosed herein. The articles can include optoelectronic devices including a display device, a solar cell, an optical data storage, a bio-probe, a carrier for drug delivery, a lamp, a LED, a LCD, a wear resistance, a laser, optical amplifier, a device for bio-imaging, optical communication, or optical computing.

Also disclosed herein are optoelectronic signaling devices comprising a composition (e.g., the solid phase compositions) as described herein. In some examples, the device can be for optical communication, optical computing, or solar energy conversion.

### Methods

Methods for enhancing upconversion luminescence of the solid phase compositions provided herein are disclosed. The methods can include irradiating the composition at a wavelength corresponding to the sensitizer absorption thereby generating a plurality of photoexcited sensitizers, allowing the plurality of photoexcited sensitizers to simultaneously transfer their energies to a higher-energy state on the conjugated polymer, and detecting luminescence in a spectral region characteristic of the conjugated polymer activated by the photoexcited sensitizers.

The methods described herein have applications in fields including biomedical imaging, biomedical therapeutics and cancer treatments, optical communications, optical computing, and solar energy conversion. Accordingly, imaging methods comprising administering to a subject a composition as described herein, irradiating the composition at a wavelength corresponding to the sensitizer absorption, and detecting luminescence in a spectral region characteristic of the conjugated polymer activated by the plurality of photoexcited sensitizers are also provided.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of measurement conditions, e.g., component concentrations, temperatures, pressures and other measurement ranges and conditions that can be used to optimize the described process.

### Example 1. Polymer-Based Cooperative Energy Pooling (CEP)

In this example, the results of two distinct polymer-based CEP systems are provided, both of which improved CEP yields over the previous Rhod6G/Stilb420 CEP system. Also presented are preliminary measurements of the internal quantum yield of CEP within one of the CEP systems. Finally, femtosecond-scale transient absorption spectroscopy (TAS) data are provided, displaying the CEP energy transfer process with time-resolution to clearly observe the energy transfer from sensitizers to acceptor.

### Methods

CEP film-making procedures: All acceptor polymers were purchased from American Dye Source. HIDC was purchased from Exciton and ZnPC was purchased from Alfa Aesar. All materials were used as received. To fabricate thin films, acceptor and polymer chromophores were separately mixed into ˜30 g/L solutions in THF solvent. These solutions were blended together in a ratio of 40 parts acceptor to one part sensitizer. This blend solution was then coated onto a glass substrate using a Zehntner ZAA 2300 blade applicator with the platen at room temperature, a blade height of 75 μm and a blade speed of 99 mm s to produce films ˜250-300 nm thick with 20-25 nm rms roughness. Glass substrates were cleaned via sonication in acetone and methanol for 5 minutes each and subsequent UV-ozone treatment for 2 minutes before film deposition.

Blend films were fabricated via blade coating onto glass substrates and were optimized for maximum film thickness in order to maximize the detectable CEP emission signal. The blade coating was carried out with a blade height of 75 μm above the substrate with the blade moving at 99 mm s with the substrate at room temperature and a stock solution of ˜30 g/L. Stylus profilometry measurements determined that the films were approximately 250-300 nm in thickness with 20-25 nm rms roughness.

Spectroscopy methods: All absorption data was taken on a VWR UV-1600PC Scanning Spectrophotometer. All emission spectra were taken on a LaserStrobe spectrometer from Photon Technology International using a GL-3300 nitrogen laser and GL-302 dye laser attachment, also from Photon Technology International. Upconverted emission spectra were measured with the emission filtered by a 500 nm short-pass filter from Thorlabs, model FES0500, to prevent reflected excitation light from interfering with the measured emission signal. Laser power was measured with a 919P-003-10 thermopile sensor from Newport. Quantum yield measurements were taken in a 4P-GPS-053-SL spectralon integrating sphere from Labsphere with some homebuilt ports and sample holders coated in a diffuse reflective coating mixed according to Knighton et al.(North 4-6 (1981)). All spectra were corrected for the spectral responsivities of the systems used for data collection.

Transient absorption measurements were on the system described in Tseng et al. (Eng. Med. Biol. Soc. 2008. 30^{th} Annu. Int. Conf. IEEE 2004 (2013)). The fundamental excitation pulse was generated using an amplified Ti:sapphire laser from Spectra-Physics (Solstice, 800 nm, 1 kHz, ˜150 fs pulse FWHM, 3.5 mJ/pulse max) which excited a TOPAS-C optical parametric amplifier from Light Conversion to generate the variable-wavelength (400 or 677 nm) pump pulse used in the experiment. The white light probe light was generated via a portion of the Ti:sapphire beam impinging upon a sapphire plate, the output of which was split into a probe and a reference beam. The pump pulses were passed through a depolarizer and chopped by a synchronized chopper to 500 Hz before reaching the sample. The pump and probe beams were focused to overlap on the sample. The transmitted probe and reference beams were coupled into optical fibers and sent to multichannel spectrometers with CMOS sensors with 1 kHz detection rates where the reference signal was used to correct the probe signal for pulse-to-pulse fluctuations in the white-light continuum. The time delay between the pump and probe pulses was controlled by a motorized delay stage. All experiments were conducted at room temperature. The change in absorbance signal (ΔOD) was calculated from the intensities of sequential probe pulses with and without the pump pulse excitation. All data was measured at using randomized time points, meaning that the data was not taken in sequential time steps in order to avoid any artifacts resulting from beam damage to the sample over time. Each spectrum was taken in less than two minutes of time in order to minimize sample burning from beam exposure, and every spectrum was measured at 10 different locations on the film and averaged together afterwards to improve signal-to-noise. ZnPC spectra were taken at 0.1 mJ/pulse excitation intensity while ADS128 and CEP film spectra were taken with at 25 μJ excitation intensity, all with a beam spot of ˜200 μm diameter. All data was corrected for chirp in the excitation pulse and any variance of T0 between measurements.

### Results

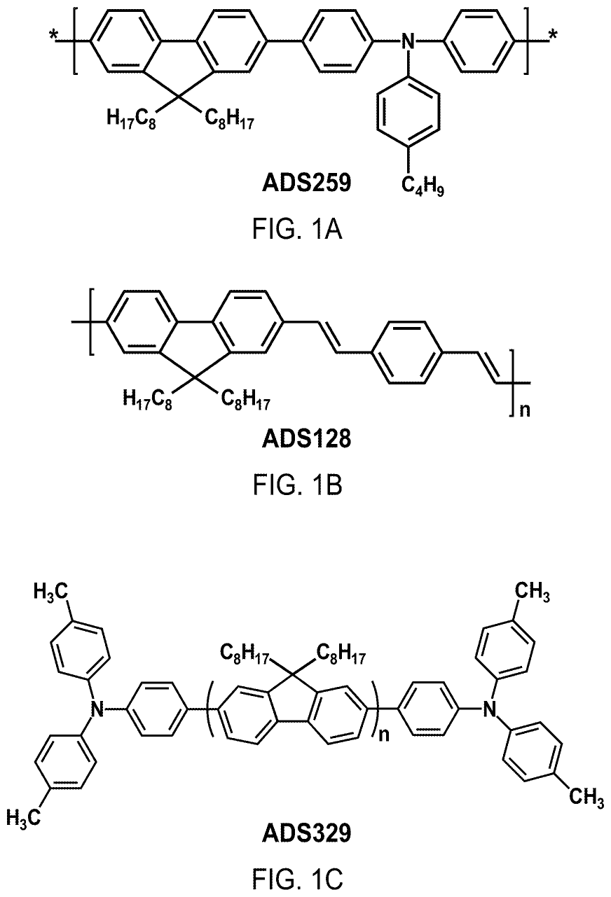

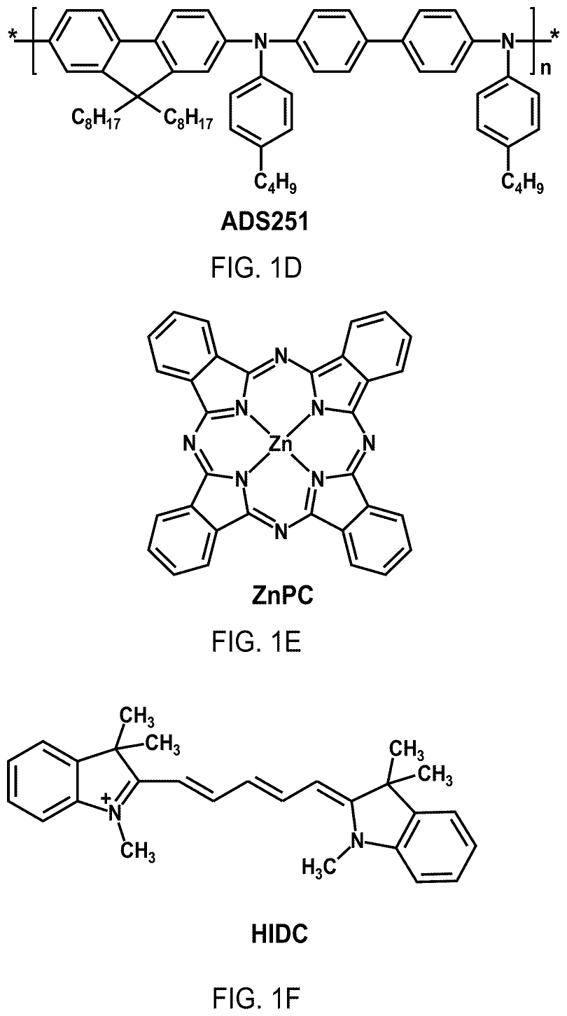

Spectral properties of cooperative energy pooling polymer systems: Since the 2PA spectrum of the acceptor determines the emission properties required of the sensitizer, a step in making the present CEP system was to identify strong 2PA acceptors. The fluorene moiety has good 2PA properties, and polymers incorporating fluorene derivatives have impressively large 2PA cross-sections. Four variations of fluorene polymers and co-polymers (displayed in FIG. 1A-FIG. 1D) were selected as candidates due to their potential for strong 2PA in the wavelength range of interest. These polymers were purchased from American Dye Source and used as received to make solutions in THF solvent. The 2PA cross-section of these molecules was measured using the two-photon excitation fluorescence method.

For most potential applications of CEP, it is desirable that the upconverted wavelength be in the near-IR range. The strong 2PA cross-section and extension into the near-IR made the ADS128 and ADS259 polymers particularly appealing as acceptors for CEP. Combinatorial testing of these polymers in blend films with various NIR dyes revealed optimized CEP upconversion yields in pairings of ADS128 with zinc phthalocyanine (ZnPC) and ADS259 with 1,1,3,3,3,3-Hexamethyl-indodicarbocyanine iodide (HIDC). The structures of ZnPC and HIDC are displayed in FIG. 1E and FIG. 1F, respectively.

Blend films were fabricated following the recipe in the methods section. The sensitizer-acceptor blend ratio is another key factor in optimizing the CEP emission signal. Films of various sensitizer/acceptor blend ratios were prepared, with a 1:40 sensitizer/acceptor ratio producing the largest upconverted signal. This blend ratio indicates that both ZnPC and HIDC exhibit strong aggregation-induced self-quenching and require low concentrations in order to maintain excited-state lifetimes long enough for effective CEP to occur. There is mention in the literature of strong aggregation-induced non-radiative decay in ZnPC, further validating this claim.

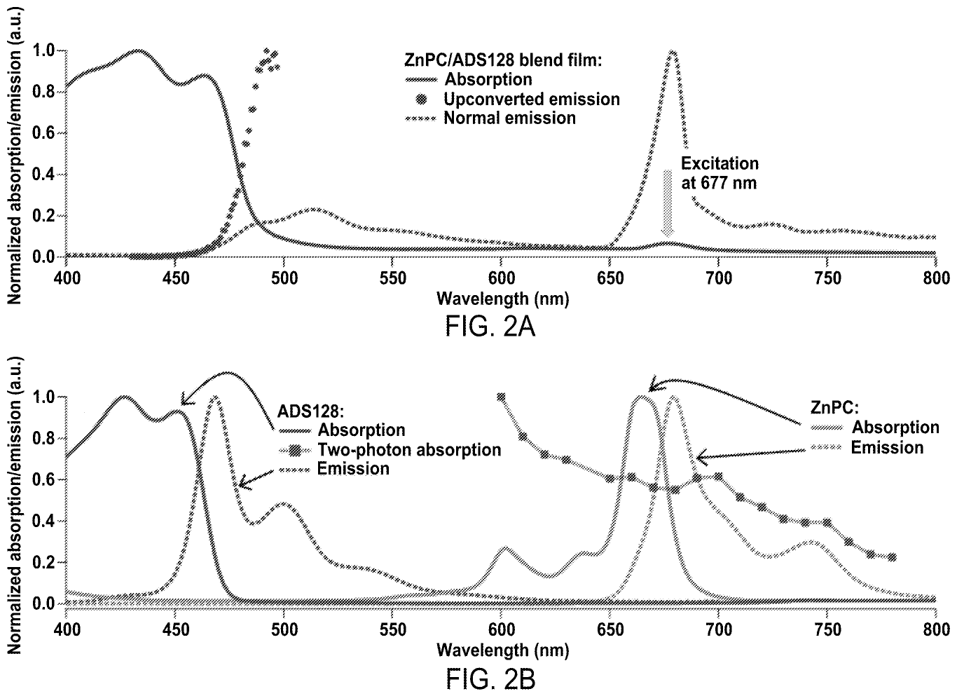

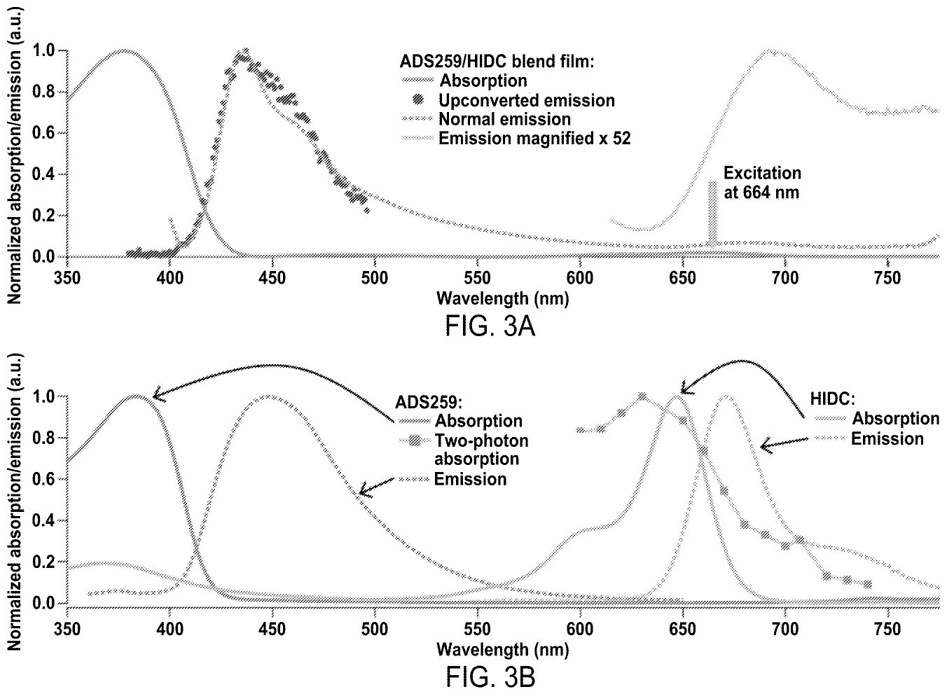

The absorption and emission spectra of the two CEP blend films, displayed in FIG. 2A-FIG. 2B and FIG. 3A-FIG. 3B, exhibit all of the features of CEP. Excitation at a wavelength corresponding to sensitizer absorption results in upconverted emission with a spectrum corresponding to that of the acceptor chromophore. In both systems the 2PA spectrum of the acceptor overlaps significantly with the emission spectrum of the sensitizer, corresponding to efficient coupling between the sensitizer transition dipole and the acceptor 2PA tensor, μ^{0a(S)} and α^{b0(A)} in Equation 1,

$${Γ}_{CEP} = {\sum }_{j , l , m , n = i}^{3} \frac{2 π}{ℏ} {\vert {μ}_{j}^{0 a ( S )} {V}_{ji} ( k , {R}^{″} ) {α}_{lm}^{b 0 ( A )} ( - k , - k ) {V}_{mn} ( k , {R}^{′} ) {μ}_{n}^{0 a ( {S}^{′} )} \vert}^{2} \qquad Equation 1$$

and hence a large CEP rate. Additionally, both of these polymer CEP systems exhibit minimal overlap between the acceptor emission spectrum and the sensitizer absorption spectrum, indicating that energy loss due to FRET from acceptor to sensitizer will play a minimal role. Excitation of the pristine acceptor at the same wavelengths as that used for CEP film excitation resulted in minimal upconverted emission, less than one tenth the emission of the corresponding CEP blend films when excited at the same wavelength, indicating that the CEP process is responsible for the vast majority of the observed upconversion.

Excitation dependence and quantum yield: The excitation dependence of upconverted emission can be a strong indicator of the efficiency of the upconversion process. The turnover point in an excitation dependence graph, namely where the excitation dependence transitions from being quadratically dependent on excitation intensity to linearly dependent, is an indicator of what excitation intensities are needed for the upconversion process to run most efficiently. A quadratic dependence on excitation intensity indicates that energy pathways other than upconversion are dominant, and hence that much of the absorbed energy is being lost to other energetic pathways before being upconverted. Conversely, linear upconversion dependence on excitation intensity indicates both an improved efficiency of upconversion as well as a constant internal quantum yield of upconversion.

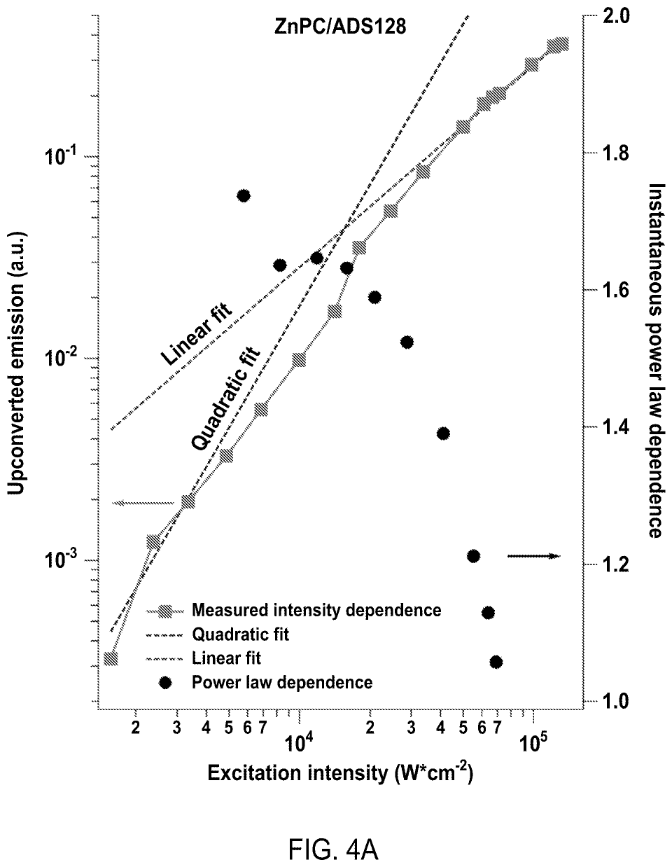

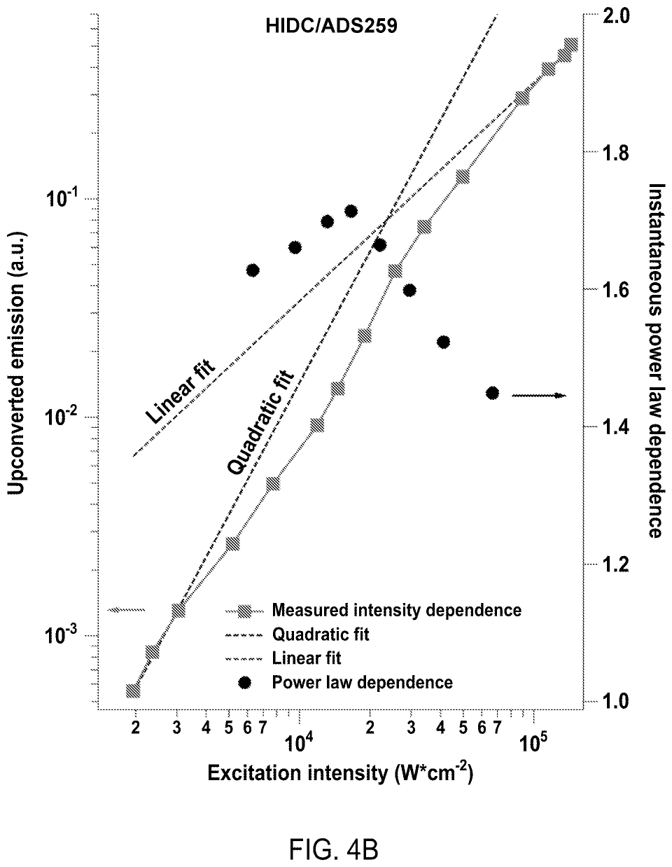

Both CEP systems measured show clear transitions from (near-)quadratic excitation dependence towards linear dependence over the two orders of magnitude range in excitation intensity measured (FIG. 4A-FIG. 4B). However, the onset and gradient of these transitions is noticeably different. The HIDC/ADS259 system exhibits a relatively small change in power law dependence and the gradient of the power law dependence as a function of excitation intensity is relatively shallow. On the other hand, the ZnPC/ADS128 system exhibits a clearer transition in power law dependence, appears to begin the transition at a lower excitation intensity, and has a steeper gradient in this transition that allows the system to reach near-linear excitation dependence at lower total excitation intensities. Both of the CEP films measured in this example exhibit excitation intensity dependencies that show improved CEP upconversion over that of Rhod6G/Stilb420 system.

In summary, an IQY lower-bound of 0.0001% for the ZnPC/ADS128 CEP system have been measured.

Time-resolved transient absorption measurements: Transient Absorption Spectroscopy (TAS) is a powerful tool capable of measuring the unique “fingerprint” of a material by detecting changes in the excited- and ground-state-absorption spectra of the material as a function of time after an excitation pulse. This change in optical density, or ΔOD, is the transient absorption (TA) signal that allows for the identification of distinct excited species within a sample based both on their spectral properties as well as their decay lifetimes as described by Berera et al. In the case of CEP, TAS provides an opportunity to directly observe the excitation of the sensitizer chromophore and follow the energy transfer to the acceptor over time after the initial excitation pulse. This type of measurement provides not only direct evidence for CEP energy transfer upconversion but also indicates the time-scales on which CEP operates.

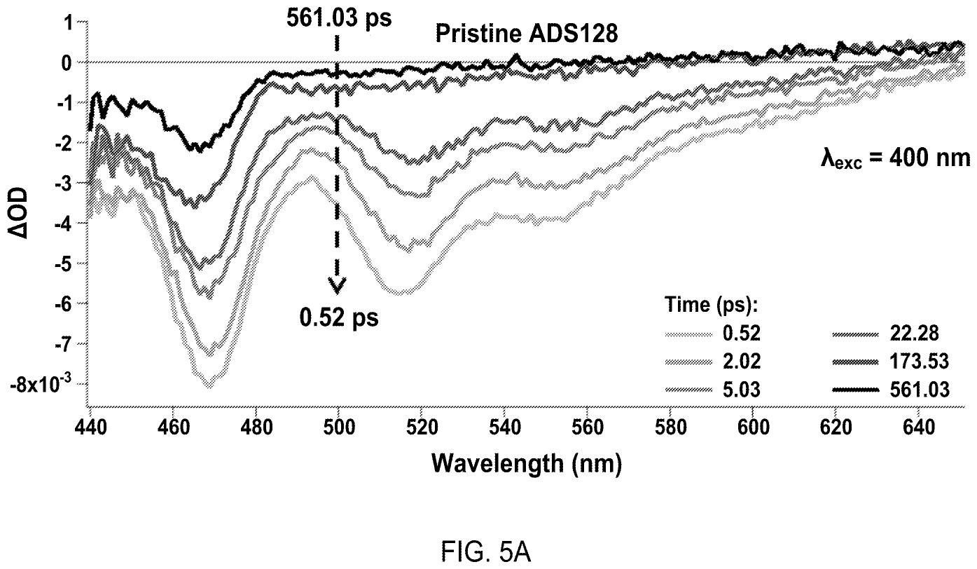

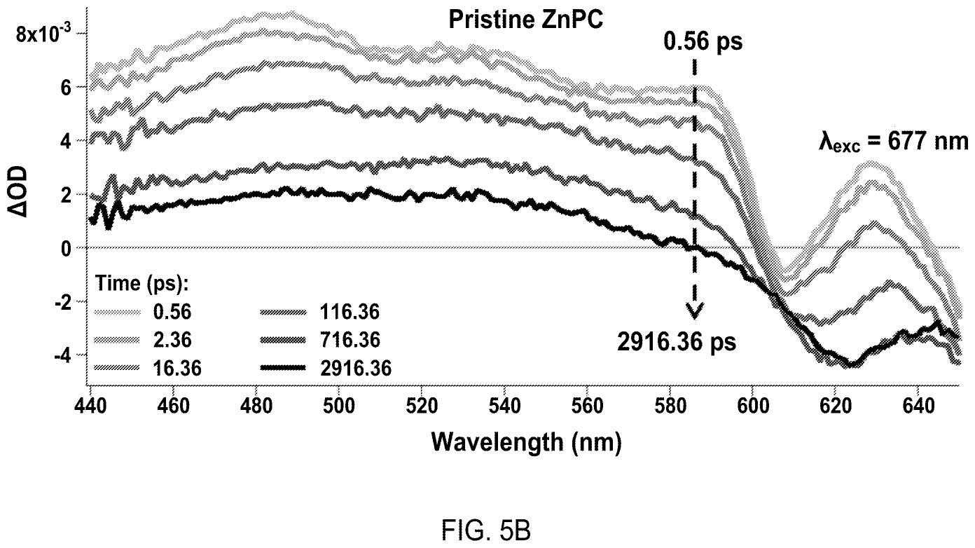

To identify the characteristic spectra of the sensitizer and acceptor chromophores, transient absorption measurements were taken of pristine films of ZnPC and ADS128. As is visible in FIG. 5A-FIG. 5B, ADS128 has uniquely identifiable features centered at 469 nm and 515 nm, with a slight shoulder feature at 555 nm, all of which have −ΔOD signals. The shape of the spectra changes over time, as noticeable in the absence of the 515 nm and 555 nm features after ˜100 ps, indicating that each of the features in the transient signal of the acceptor have distinct decay rates. While a more thorough analysis of these features might yield assignments to the various electronic and vibrational modes of the acceptor molecule, this type of analysis would reveal more about the behavior and characteristics of the ADS128 molecule itself than it would about the CEP process and hence is extraneous to this example. The primary concern was identifying the process of CEP energy transfer from the sensitizer (ZnPC) to the acceptor (ADS128), and since the ADS128 acceptor has distinctive transient features in its TA spectrum, an analysis that is essentially binary can be conducted: if the blend film exhibits transient features matching those of the ADS128 spectrum then it was concluded that there exists a population of acceptors in the excited state and vice versa.

The TA spectrum of ZnPC (FIG. 5A-FIG. 5B) has a broad plateau extending from ˜430-600 nm with a +ΔOD signal that is composed to sub-features at 485 nm, 530 nm, and 596 nm, each with distinct decay rates and matching similar data in the literature. The feature at 596 nm has a noticeably faster rise and decay time than the other features, further complicating any lifetime analysis.

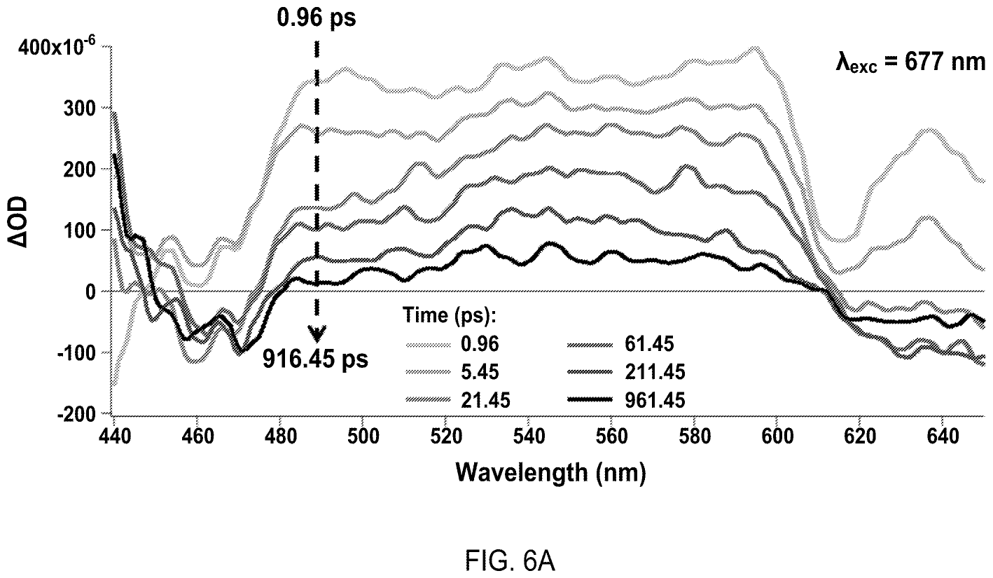

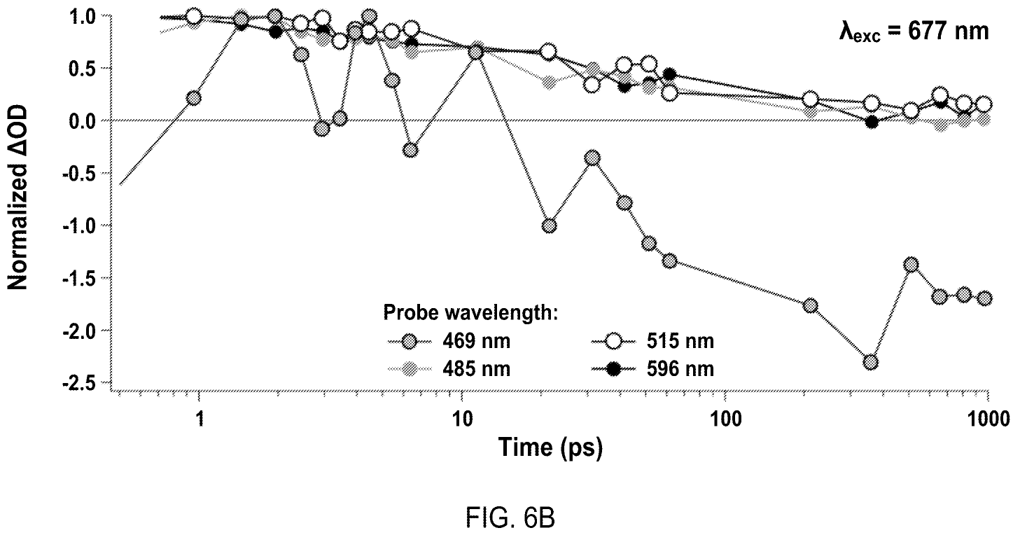

While the acceptor and sensitizer chromophores have distinct and uniquely identifiable features, the fact that the main features of ADS128 and ZnPC overlap in wavelength, have opposite ΔOD signals, and have distinct lifetimes indicates that the signal from the CEP blend film will be a complex superposition of the two signals as a function of time. As expected, the TA signal from the CEP blend film (FIG. 6A-FIG. 6B) does appear to contain components from both sensitizer and acceptor TA spectra. The CEP blend film exhibits a clear plateau extending from 485-590 nm that corresponds to a similar feature in the sensitizer spectrum, as well as a dip centering around 469 nm that corresponds to the acceptor signal peak.

Analyzing the TA signal of the CEP blend film is somewhat complex due to the myriad energetic processes occurring within and among each of the chromophore types. As discussed above, the goal of this analysis is to characterize the process of CEP energy transfer from sensitizer to acceptor. Keeping this in mind, analysis of TA signals that are relevant primarily to the internal processes within a chromophore (i.e. the various peaks and associated lifetimes in the sensitizer or acceptor spectra) as well as signals related to processes that occur after the CEP process (i.e. any evolution of the signal components corresponding to the acceptor after its initial excitation) are discussed. What remains is the evolution of the sensitizer signal after excitation and its subsequent energy loss processes (both CEP and various internal decay processes) as well as the growth of the acceptor signal due to CEP energy transfer from the sensitizer.

The ability to accurately identify the various features present in the TA data is necessary so that they may be properly assigned to their sources. This is made somewhat easier by the fact that in the range of interest (˜440-600 nm) the acceptor signal is entirely −ΔOD while the sensitizer signal is entirely +ΔOD.

While the features at 485 nm, 515 nm, and 596 nm have distinct lifetimes in the pristine films, they appear in the blend film (FIG. 6B) with nearly identical decay lifetimes. The sensitizer TA spectrum exhibits roughly similar behavior at each of these wavelengths, suggesting that these wavelengths (as well as the entirety of the plateau of the signal) correspond to the sensitizer excited state.

The 469 nm feature's kinetic trace has a delayed rise compared to the others with a subsequent small rise to +ΔOD values. Since the kinetic trace of the acceptor at 469 nm never exhibits a +ΔOD signal, this rise was attributed to excitation in the sensitizer. Since the anomalous ultrafast signal in ADS128 is entirely absent after 200 fs and the ZnPC signal is entirely +ΔOD, all −ΔOD signal at 469 nm after ˜1 ps can be attributed to the main TA feature of the acceptor, and hence to acceptor states excited by the CEP process. After 1 ps, this 469 nm feature proceeds to decrease at a much faster rate than the other features and after a few 10 s of picoseconds exhibits a −ΔOD signal.

The negative value of this 469 nm feature is significant because it allows positive identification of this feature as corresponding to the excited acceptor. The ZnPC TA signal maintains a relatively uniform +ΔOD value throughout its entire decay lifetime, which suggests that any deviation from this flat, positive signal is due to excited acceptor. However, deviation from a flat signal would not be conclusive proof of excited acceptor states. A hypothetical +ΔOD signal at 469 nm that had reduced OD compared to the rest of the plateau signal at longer wavelengths could potentially be caused by a change of shape of the sensitizer signal when in a blend film. Evolution of this hypothetical feature towards reduced, but still positive, OD could potentially indicate either increased acceptor excitation or simple decay of sensitizer excitation without the possibility of distinguishing between the two.

However, the feature at 469 nm is negative and since the sensitizer signal has no −ΔOD components at any point in time it would be impossible for the TA signal to exhibit −ΔOD without the presence of excited acceptor chromophores. The −ΔOD may be either due solely to excited acceptors or due to a superposition of positive signal from excited sensitizers and a stronger negative signal from excited acceptor states, but either interpretation indicates that the acceptor has successfully been excited and hence CEP must have occurred. While the actual TA spectrum in the 450-480 nm range appears to be quite noisy, it must be noted that the signals in that wavelength range in both the component chromophore spectra are quite clear, indicating that the noisy, near-zero signal is not due to a lack of signal but rather due to a super-position of positive and negative ΔOD signals. While the absolute magnitude of the −ΔOD signal is quite small, this signal was averaged over ten distinct measurements at different locations on the film and is also consistently negative with no signs of decay out to 1 ns, and thus is not an artifact of noise in the data.

Simultaneous excitation of the acceptor and sensitizer could yield an increasingly −ΔOD only if the +ΔOD component of the signal (sensitizer) decayed more rapidly than the −ΔOD component (acceptor), leaving an overall −ΔOD signal after sensitizer decay. However, the TA spectra of the pristine samples indicates that the acceptor has a distinctly longer lifetime than the sensitizer, indicating that the growing −ΔOD feature must be due to the acceptor becoming increasingly excited at delayed times. Since CEP requires excitation of the sensitizers followed by subsequent energy transfer to the acceptor, this delayed excitation evident in the data is further evidence for CEP.

Analyzing the actual kinetics of CEP in this system is somewhat complex due to the superposition of the sensitizer signal decay with both the prompt and delayed rise and subsequent decay of the acceptor signal at the same wavelength. Thus, while it can be concluded that the delayed rise of a −ΔOD peak could only occur in the presence CEP, the actual rate of CEP cannot be deconvolved from the other overlapping processes occurring simultaneously and hence the data cannot be fitted to extract a CEP rate.

Subtracting the signal at 469 nm from the average signal of the plateau region (490-590 nm) would yield a signal weighted by the numbers of excited sensitizers versus acceptors. Without the ability to accurately correct for that weighting the rate of change of the 469 nm feature would be a convolution of the sensitizer decay and the CEP rate, once again preventing the extraction of an actual rate of the CEP process.

After excluding all the methods of extracting rates of the CEP process, the remaining pathway forward is a general estimate of the timescale on which CEP occurs. Since the feature at 469 nm begins its negative slope in the 1-10 ps timescale and flattens out by ˜500 ps, it can be estimated that the timescale of CEP in this system is in the range of tens-to-hundreds of picoseconds.

### Discussion

Despite the lack of detailed lifetime analysis of the TA data, it was observed that the acceptor signal rises to full −ΔOD strength within ˜400 ps and then sustains with minimal decay beyond 1 ns.

Considering that the pristine acceptor signal decayed by a factor of two within ˜150 ps, the endurance of the −ΔOD signal at 469 nm past 1 ns in the blend film is notable and potentially indicates excited acceptor states with lifetimes longer than hundreds of picoseconds. It is possible that the anomalously long lifetime of the CEP-excited state is due to morphological selectivity of the CEP process. For instance, CEP is more likely to occur when there is a minimal separation distance between the sensitizers and the acceptor, and hence CEP energy transfer is likely to preferentially occur to acceptor chromophores whose nearest neighbors are sensitizers rather than other acceptors. This isolation from other acceptors then potentially extends the lifetime of the CEP-excited state towards its inherent radiative lifetime by reducing pathways for non-radiative decay via self-quenching. Thus, heterogeneity of the CEP film morphology may play an important role in the CEP rates and excited state lifetimes. While it is possible that the persistence of the excited acceptor signal may indicate the transfer of energy to a longer-lived state in the acceptor, such as a triplet state, it is unlikely that such a state would exhibit a TA feature at an identical wavelength to that of the first excited singlet state. Further investigation into the excited state lifetimes of the particular chromophores that are the most likely to undergo CEP may yield insight into the role of local morphology on CEP rates.

Even without exact values for the rate of CEP in this system one can still use the approximate CEP rate to find an approximate sensitizer-acceptor chromophore separation distance in the ADS128/ZnPC system. Using literature values for ZnPC of τ_{rad}=4.1 ns and Φ=0.28 and combining these with the peak emission wavelength of ZnPC of λ=677 nm and the approximate 2PA strength of the acceptor σ₂˜4*10⁵ GM (approximating 2PA cross-section values using an analogous polymer acceptor studied by Wu et al.), it was found that the sensitizer-acceptor separation distance in this system is estimated to be between 2.3-3.2 nm. This is an entirely reasonable range of chromophore separation in a thin film, especially when considering that while the ZnPC chromophores are only 1-2 nm in width and much thinner in cross-section, one would expect to find them at somewhat greater (average) distances from the acceptor chromophores due to the low concentration of sensitizer chromophores in the CEP blend film. Further adjustments to film morphology could potentially reduce this separation distance and increase the overall rate and yield of CEP upconversion.

The overall results of this work indicate a step forward in the development of CEP systems. While the improved upconversion rates compared to the previous Stilb420/Rhod6G system allowed for more advanced measurements techniques such as IQY and TA, there is still plenty of room for improvement. The low upconversion rates left yielded very small signals with high levels of noise, which increased the uncertainty in the data and reduced the extent of the analysis that was able to be carried out. Higher signal-to-noise ratios would allow for improved measurements of IQY and better lifetime fits to the TA spectra.

The systems in this work have large acceptor 2PA cross-sections, 2PA that extends to longer wavelengths, improved spectral overlap between acceptor and sensitizer, and reduced FRET energy loss pathways, all of which are factors that are expected to improve CEP rates. Thus, an exact mechanism contributing to improved CEP over the previous systems was not made.

One aspect of the CEP system in these polymer films was the self-quenching behavior of the sensitizer. The fact that this blend ratio exhibited the highest CEP emission indicates that sensitizer self-quenching remains a dominant energy loss mechanism in these polymer CEP systems. The use of sensitizers with high QY even when aggregated would directly increase both the CEP rate but also the CEP radius. The isolation of sensitizer chromophores in these systems also drastically reduces the diffusivity of sensitizer exciton. An overly small diffusivity will result in minimal CEP yields.

High self-quenching the sensitizer chromophore leads to reduced CEP radius, reduced overall absorbance of the blend film, and reduced exciton diffusivity, all of which non-linearly reduce overall CEP yields. Therefore, the fact that any CEP upconversion was observed with such lossy sensitizers indicates that with even slightly improved sensitizers potentially dramatic improvements to CEP efficiency may result.

### CONCLUSION

In this example was presented two new CEP systems, both based on polymeric acceptors. These systems exhibited excitation intensity dependencies that show a clear transition from quadratic to linear, with the AD128/ZnPC system nearly approaching the linear regime. Internal quantum yield measurements on the ADS128/ZnPC system indicated minimum efficiency value of 0.0001%, but a number of factors indicate that the true efficiency may be higher than this. Transient absorption measurements on this same system revealed delayed excitation of the acceptor when excited at sensitizer absorption wavelengths, and displayed CEP occurring on a timescale of tens to hundreds of picoseconds. Further improvements to the CEP system, particularly the sensitizer chromophore, are expected to yield improved results and allow for more in-depth investigation utilizing advanced spectroscopies.

Publications cited herein are hereby specifically incorporated by reference in their entireties and at least for the material for which they are cited.

While it should be understood that while the present disclosure has been provided in detail with respect to certain illustrative and specific aspects thereof, it should not be considered limited to such, as numerous modifications are possible without departing from the broad spirit and scope of the present disclosure as defined in the appended claims. It is, therefore, intended that the appended claims cover all such equivalent variations as fall within the true spirit and scope of the invention.

1. A method for enhancing upconversion luminescence of a solid phase composition comprising a multi-photon absorbing conjugated polymer and a sensitizer, wherein the conjugated polymer is separated from the sensitizer by an average distance of 5 nm or less, and wherein the molar ratio of the sensitizer to the conjugated polymer is from 1:10 to 1:100, the method comprising:
irradiating the composition at a wavelength corresponding to the sensitizer absorption thereby generating a plurality of photoexcited sensitizers,
allowing the plurality of photoexcited sensitizers to simultaneously transfer their energies to a higher-energy state on the conjugated polymer, wherein the emission spectrum of the photoexcited sensitizer at least partially overlaps with the multi-photon absorption spectrum of the conjugated polymer, such that there is resonant coupling between the sensitizer transition dipole and the conjugated polymer multi-photon tensor, and
detecting luminescence in a spectral region characteristic of the conjugated polymer activated by the photoexcited sensitizers.

2. The method of claim 1, wherein the multi-photon absorbing conjugated polymer is a two-photon absorbing conjugated polymer.

3. The method of claim 1, wherein the emission spectrum of the conjugated polymer exhibits negligible overlap with the absorption spectrum of the sensitizer.

4. The method of claim 1, wherein the conjugated polymer comprises a polyfluorene, a polyarylene, a polyphenylene, a polyanthracene, a polypyrene, a phenanthrene, a heterocyclic polyarylene, a poly(pyridine), an oxadiazole-containing polymer, a quinoline-containing polymer, a silole-containing polymer, a poly(3-alkyl-thiophene), or a combination thereof.

5. The method of claim 1, wherein the conjugated polymer comprises a polyfluorene.

6. The method of claim 1, wherein the conjugated polymer comprises a polyfluorene selected from the group consisting of: 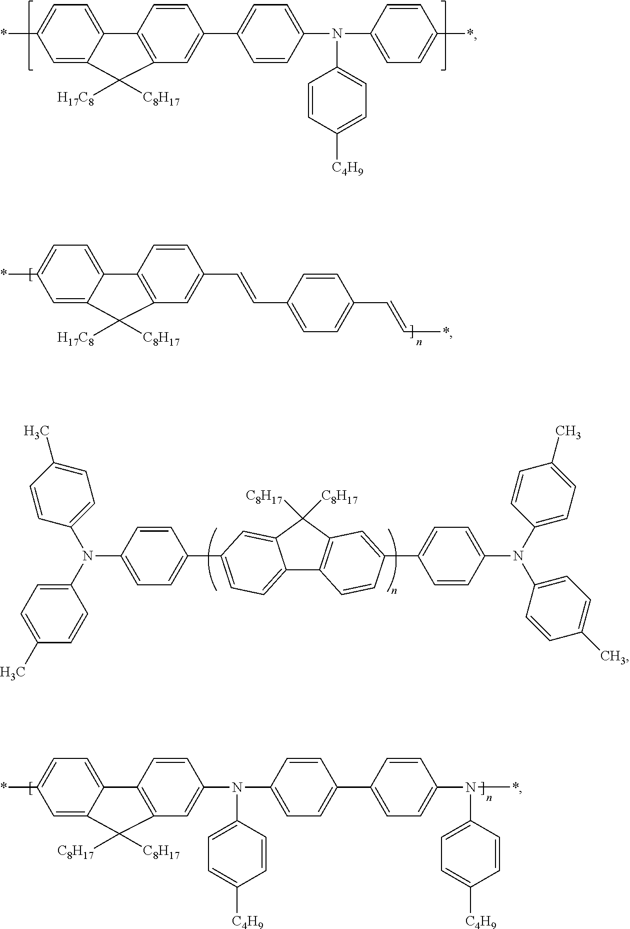
and combinations thereof.

7. The method of claim 1, wherein the sensitizer comprises a near infrared absorbing organic chromophore.

8. The method of claim 1, wherein the sensitizer comprises a cationic dye, an anionic dye, a nonionic dye, an amphoteric dye, a metal-ligand complex, fluorescein, chlorophyll, a phthalocyanine, an indodicarbocyanine, or a mixture thereof.

9. The method of claim 1, wherein the sensitizer comprises: 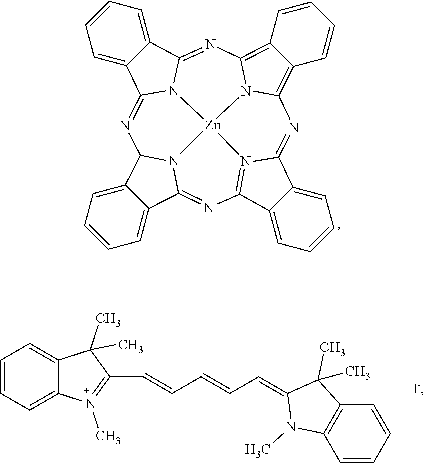
or a combination thereof.

10. (canceled)

11. (canceled)

12. The method of claim 1, wherein:
the composition is a nanofilm having an average thickness of 500 nm or less; or
the composition comprises nanoparticles having an average particle size of 500 nm or less.

13-30. (canceled)

31. A composition for enhancing upconversion luminescence comprising:
a solid phase composition comprising multi-photon absorbing conjugated polymer and a sensitizer, wherein the solid phase composition is in the form of a nanofilm or nanoparticles, and wherein the conjugated polymer is separated from the sensitizer by an average distance of 5 nm or less,
wherein the emission spectrum of the sensitizer at least partially overlaps with the multi-photon absorption spectrum of the conjugated polymer, such that when the sensitizer becomes electronically excited, there is resonant coupling between the sensitizer transition dipole and the conjugated polymer multi-photon tensor, and
wherein the sensitizer and the multi-photon absorbing conjugated polymer are in a molar ratio from 1:10 to 1:100.

32. (canceled)

33. The composition of claim 31, wherein the multi-photon absorbing conjugated polymer is a two-photon absorbing conjugated polymer.

34. The composition of claim 1, wherein the emission spectrum of the conjugated polymer exhibits negligible overlap with the absorption spectrum of the sensitizer.

35. The composition of claim 31, wherein the conjugated polymer comprises a polyfluorene, a polyarylene, a polyphenylene, a polyanthracene, a polypyrene, a phenanthrene, a heterocyclic polyarylene, a poly(pyridine), an oxadiazole-containing polymer, a quinoline-containing polymer, a silole-containing polymer, a poly(3-alkyl-thiophene), or a combination thereof.

36. The composition of claim 31, wherein the conjugated polymer comprises a polyfluorene.

37. The composition of claim 31, wherein the conjugated polymer comprises a polyfluorene selected from the group consisting of: 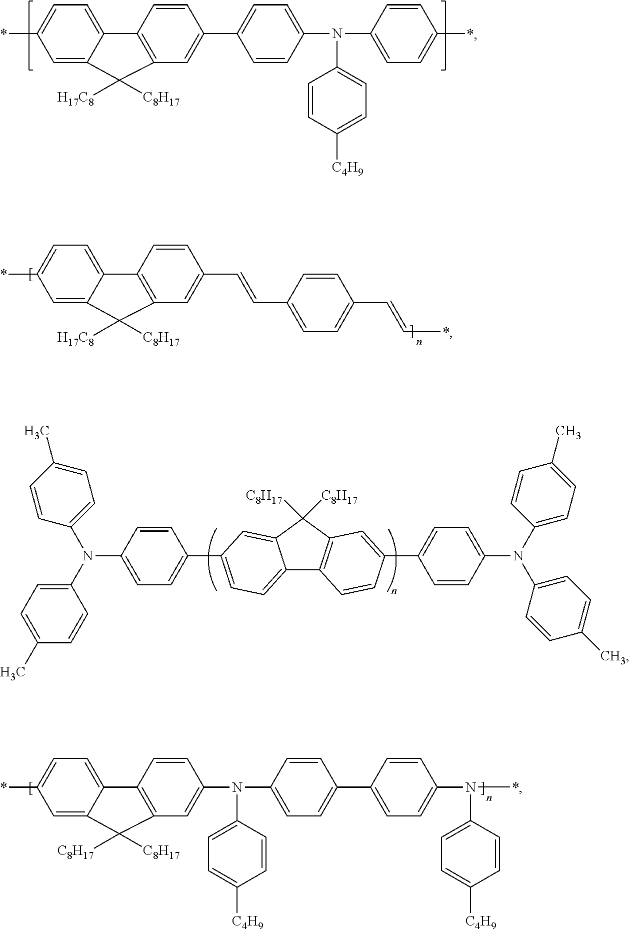
and combinations thereof.

38. The composition of claim 31, wherein the sensitizer comprises a near infrared absorbing organic chromophore.

39. The composition of claim 31, wherein the sensitizer is selected from a cationic dye, an anionic dye, a nonionic dye, an amphoteric dye, a metal-ligand complex, fluorescein, chlorophyll, a phthalocyanine, an indodicarbocyanine, or a mixture thereof.

40. The composition of claim 31, wherein the sensitizer comprises: 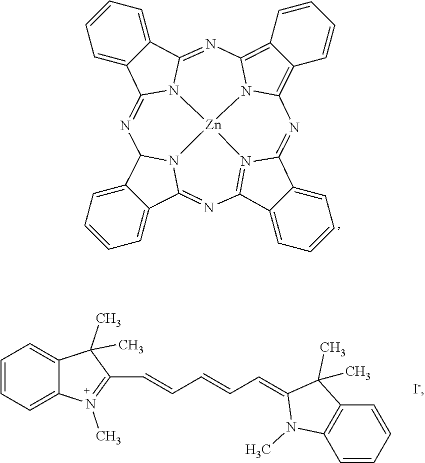
or a combination thereof.

41. The composition of claim 31, wherein:
the composition is a nanofilm having an average thickness of 500 nm or less; or
the composition comprises nanoparticles having an average particle size of 500 nm or less.

42-46. (canceled)
